(19) United States
(12) Patent Application Publication
(10) Pub. No.: US 2024/0358467 A1
(43) Pub. Date: Oct. 31, 2024

(54) MANAGEMENT SYSTEMS FOR SURGICAL INSTRUMENTS AND ITEMS USED DURING SURGERY

(71) Applicant: Sure Count Surgical, LLC, Los Angeles, CA (US)

(72) Inventor: Lawrence M. Richman, MD, Los Angeles, CA (US)

(21) Appl. No.: 18/739,014

(22) Filed: Jun. 10, 2024

Related U.S. Application Data
(63) Continuation of application No. 18/140,505, filed on Apr. 27, 2023, now Pat. No. 12,036,050.

(51) Int. Cl. A61B 50/33; A61B 90/00

(52) U.S. Cl. CPC A61B 50/33; A61B 2090/0804; A61B 2090/0807

(57) ABSTRACT

The management system mounts rear upright instrument supports on the base of a tray. Each rear support holds the rear end of instruments such as forceps or scissors with the finger holes spaced-apart. The forward end of the instruments is held by a forward support. That support has a bar over the instruments, which is positioned to be horizontal when the forward support (and the rear support) is full.

### RELATED APPLICATIONS

This is a continuation application that claims priority to U.S. patent application Ser. No. 18/140,505, filed Apr. 27, 2023, and entitled “Management Systems for Surgical Instruments and Items Used During Surgery.”

### BACKGROUND

### Field

Management systems for surgical instruments in operating rooms.

### General Background and State of the Art

Surgeons and their staff need their instruments and other items used during surgery to be readily accessible and well organized. Looking for forceps hidden by or contacting other instruments, for example, adds crucial time to a surgery. That is undesirable for the patient because delays increase the length of the surgery and excessive time under anesthesia, and it increases the risk of post-operative infection.

Increasing the time for each surgery is also detrimental to surgeons, other physicians, and staff and to the hospital or surgical center. Delays for each surgical team compound the delays for other teams, which also can delay the next procedure and limit operating room efficiency and increased patient pass through.

After each surgery, the team commonly takes all surgical instruments used during surgery or taken into the operating room and mounts them through their finger openings on a stringer, two parallel, spaced stainless steel rods. The rods (stringers) organize and hold the instruments together for easier transport and organization but fall short of individual instrument identification and ready access. The instruments are sprayed with a disinfectant that kills microbes that might otherwise float freely into the hospital corridors during transport back to the sterilization processing department as well as becoming airborne within the sterilization department itself. Loading the instruments on the rods, removing them for sterilization and then organizing them for another surgery are time-consuming, inefficient activities that increases the risk of glove punctures or dropping instruments to the floor requiring replacements.

Increased costs and decreased efficiency are not the only concerns. Each team must account for all instruments when the procedure starts and ends. As an illustration, if the team starts with a ten of one instrument and uses all but three, they must account for the seven used and the three unused. If any are missing, the team must determine how many instruments are missing. Once known, locating one or more missing instruments may be difficult and time consuming, all of which adds up to the duration of the procedure especially when accounting for each surgery, each operating room, each hospital, and hospitals in general. Some estimates assume $40/minute for each operating room delay.

The managed instruments must be sterile for use during surgery, and particular attention must be directed to observing instruments from coming into contact with non-sterile areas or objects in the operating room. This management system facilitates sterilization of instruments, organizes them together and diminishes inadvertent contamination in the operating room. Doing so is more efficient than sterilizing the instruments separately and then loading the instruments for each upcoming surgery.

High-temperature steam used to sterilize instruments could adversely affect management systems' components, so choosing the parts of the system that the heat and substances will not damage is important.

Different surgical procedures, e.g., cardiac versus orthopedic, often require different instruments and different amounts of the same instrument. The management system greatly facilitates organization, accountability, and chain of custody of the surgical instruments.

### SUMMARY

The management system for holding surgical instruments includes a tray having a base. The tray may have sidewalls and a cover extending to the sidewalls over the base. The base has openings to let the steam and EO gas circulate around the surgical instruments. The sidewalls and cover may have similar openings.

Rear upright instrument supports attach to the base, and each support holds the handle end of a stack of surgical instruments. The rear upright instrument supports have projections that conform to structures of one end of the surgical instruments. The rear upright instrument support has at least one foot, each of which attaches to the base.

A forward support, which also is secured to the tray's base, is positioned forward of the rear upright instrument support and its stack of surgical instruments. The forward supports hold the forward part of the instruments. For many instruments, the forward support is near the instrument's hinge, but it may be spaced from the hinge especially for longer forceps or other instruments. The forward support has spaced-apart members that may be U-shaped, and each spaced-apart member has spaced-apart segments. A single member may replace the two, spaced-apart members. The other ends of the instruments rest between the forward support's members. An arm mounts on a hinge between the segments of one spaced-apart member and extends toward the segments of the other spaced-apart member. When the stack of instruments has a predetermined number of instruments, the arm contacts the top instrument and is horizontal, i.e., parallel with the tray's base.

The arm pivots upward to load the instruments and to be free of the instruments so the upper most instrument of the stack of instrument can be added or removed from the stack. Each time the top instrument is removed from the stack, the arm pivots back below the horizontal starting position to rest on what becomes the top instrument. When another instrument is removed from the stack, the arm can pivot farther.

Numbers may be printed on the forward support near the end of the arm. The number “1” is positioned where the end of the arm drops when one instrument is removed from the forward support. “2” is positioned where the end of the arm is when the second instrument is removed, and so on. The numbers would alert the operative team what instruments are in use in real time, what instrument(s) may be missing deep within a body cavity or under a drape, floor, etc. The security alert bar is restricted from descending too far out of finger reach by a horizontal metal bar between the two sidewalls.

The type of instrument can be printed on the base or on the supports.

### BRIEF DESCRIPTION OF THE DRAWINGS

FIG. 1 is a perspective view of the management system with an attached top cover.

FIG. 2 is a perspective view of the management system with a top cover released and elevated, so the internal structure is visible.

FIG. 3 is a plan view of the management system.

FIG. 4 is a perspective view of a forward support for the management system.

FIG. 5 is another perspective view of a forward support for the management system with an arm in an open position.

FIG. 6 is a perspective view of one version of the management system's rear upright instrument support holding part of the handles of an instrument.

FIG. 7 is a perspective view of another version of the management system's rear upright instrument support holding part of the handles of an instrument.

FIG. 8 is a perspective view of another version of the management system's rear upright instrument support holding part of the handles of an instrument.

FIGS. 9 and 10 are sectional views through plane 9-9 of FIG. 2 showing the system's locking mechanism.

### DETAILED DESCRIPTION

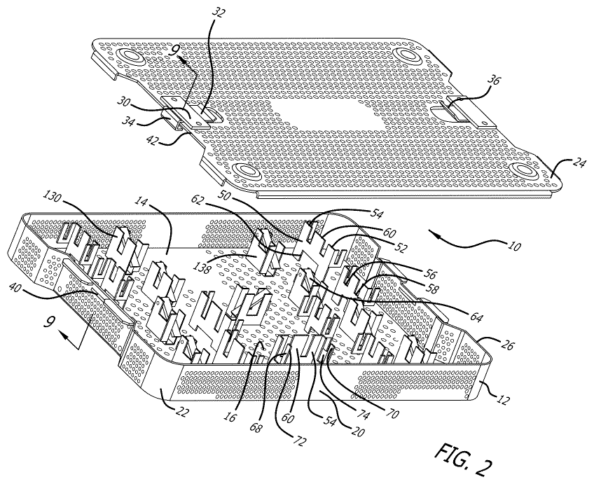

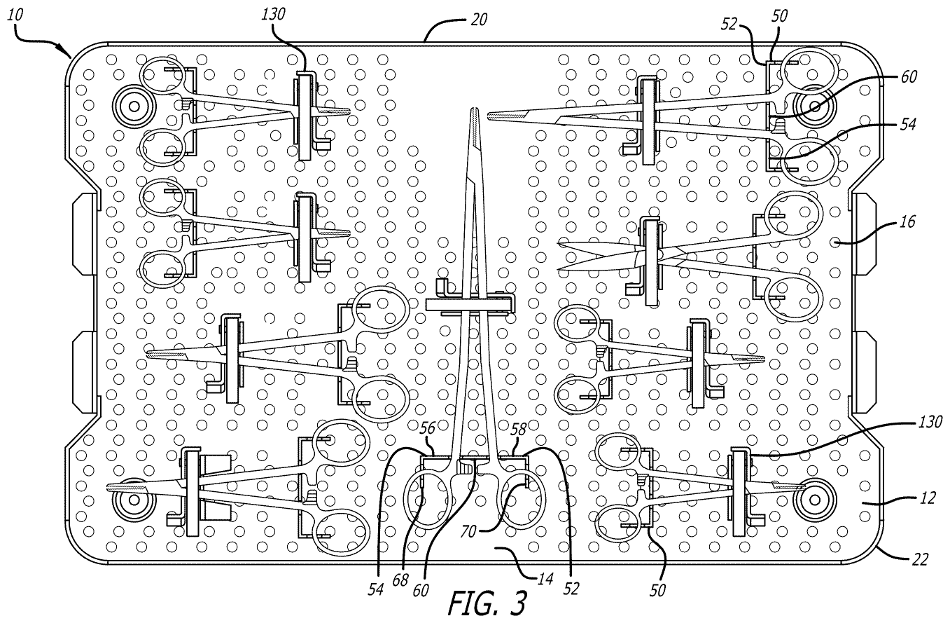

Management system 10 includes a tray 12 with a base 14 (FIGS. 2, 3). The tray has an array of openings 16, which are circular and aligned in FIGS. 2 and 3, but the openings may be other shapes, and may be arranged differently.

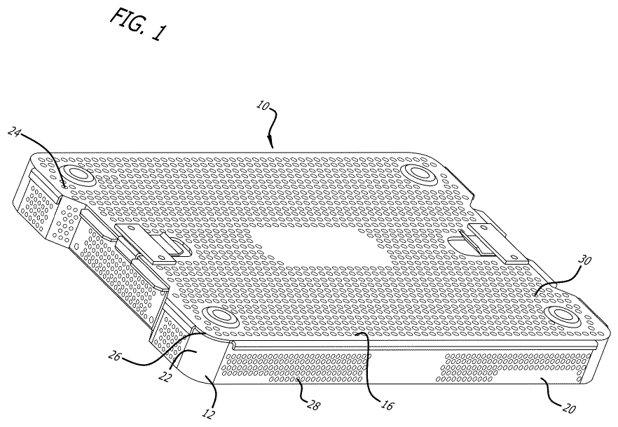

Sidewall 20 extends upright from the four side edges of base 14. The corners 22 of the sidewalls may be curved to avoid sharp corner edges. The sidewall also has an array of openings 28, which may be of a different size or shape from openings 16 in the base. Lid 24 (FIG. 1) may attach to the top 26 of the sidewall. The lid also has an array of openings 30.

Base 14 or other parts may have printing showing the instrument for each location.

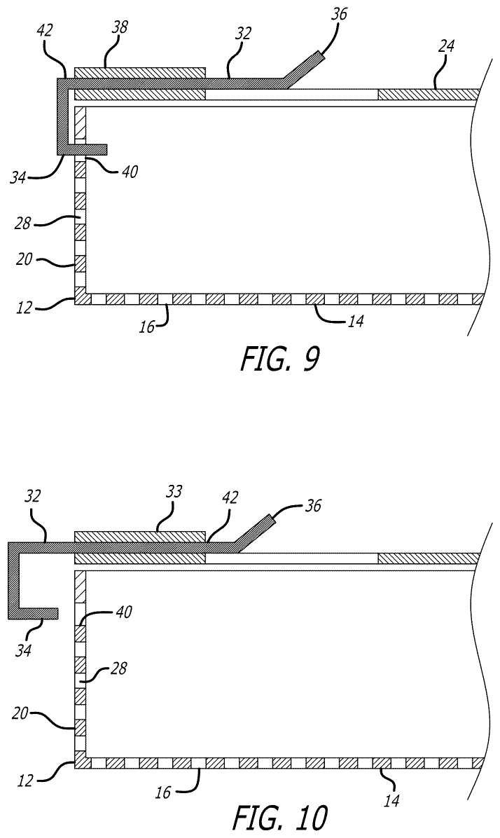

Lid 24 can be secured to tray 12. See FIGS. 1, 9, and 10. Slider 32 can move horizontally in opening 42. The slider may have a finger-gripping projection 36 and an engagement projection 34. Pulling on the slider to the right (FIGS. 9 and 10) moves the engagement projection in slot 40. Pushing the slider to the left moves the engagement projection out of the slot to release lid 24.

The tray's dimensions may vary. In one system, tray 12 may be 51.5 mm wide and 32.5 mm deep. Sidewall 20 may be 4.8 mm high. With lid 24 attached to the tray, the tray's overall dimensions are 52.5 mm wide, 33.5 mm deep and 5 mm high. The tray can fit within a wide-body Aesculap® SterilContainer. See www.aesculapusa.com (accessed Mar. 29, 2023). Steam and EO pass through openings 16, 28, and 30 and reach the instruments in the tray to sterilize them. The base, sidewall and lid may be aluminum, but they may be other non-corrosive materials that the heat and chemicals do not affect adversely during sterilization. Depending on the height of the tray including its sidewall, mounting two trays together, one above another, may fit in Aesculap containers. Other manufacturers may choose to expand the width of their sterilization containers to accommodate the present management system. If wider modules are not in stock, standard hospital procedure allows wrapping any item in CSR wrap, sterilizing the contents in the wrap, and sending it to the operating room. Depending on the instruments loaded on the tray, a fully laden tray weighs about 2% kg (≈6 lbs.).

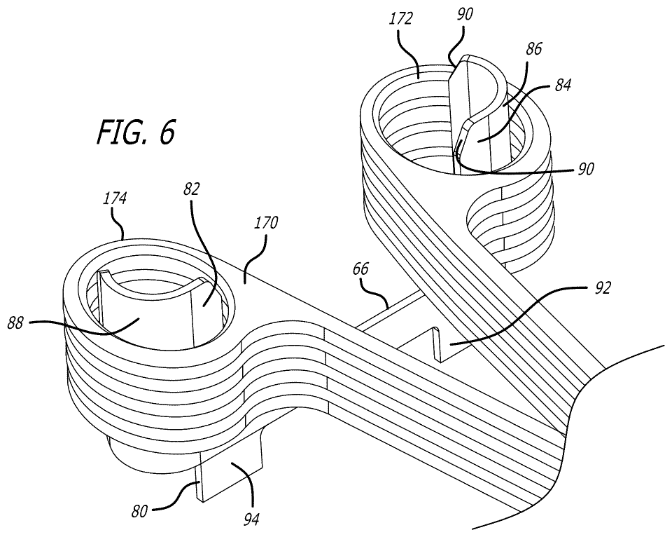

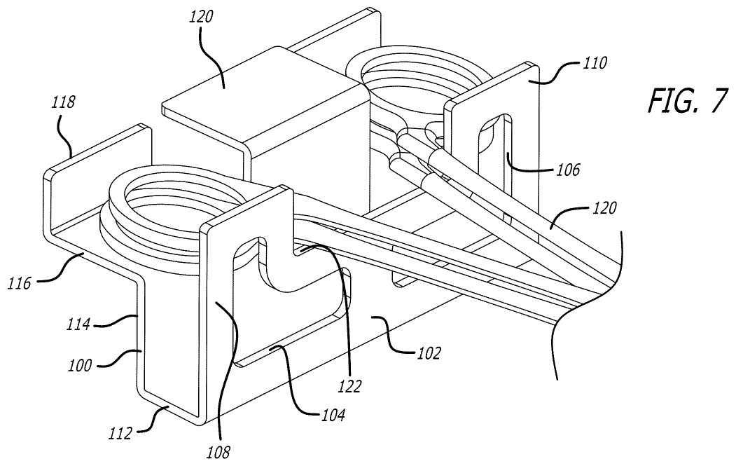

Rear upright instrument support 50 attach to base 14 for holding instruments in tray 12. See FIGS. 2, 3 and 8. See also rear upright instrument support 80 (FIG. 6) and rear upright instrument support 100 (FIG. 7). Support 50 may be stamped stainless steel or another non-corrosive material. The rear upright instrument supports may be attached to the base using automatic laser welding or other fastening techniques. Openings (not shown) may be provided in the base into which structure from the rear upright instrument supports extends for fastening.

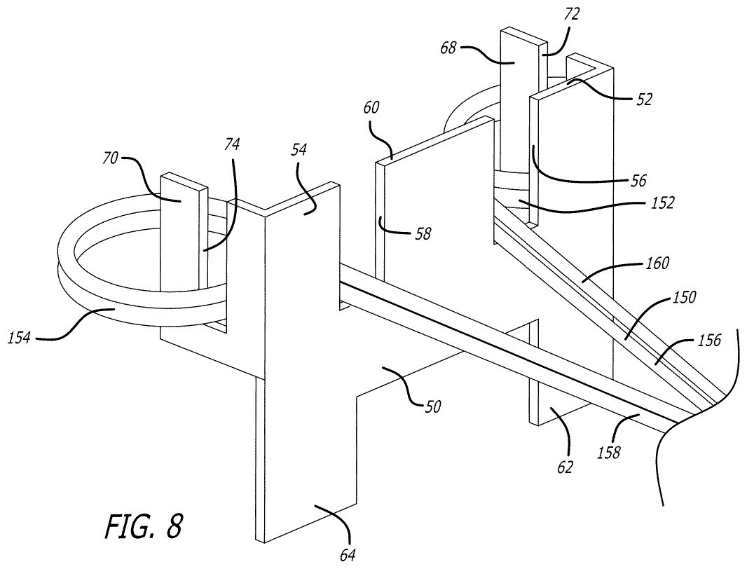

Rear upright instrument supports can have different configuration that may depend on the instruments they hold. Because of the number of rear upright instrument supports in FIGS. 2 and 3 and the distances between them, reference numbers may be on the elements of different supports. But FIG. 8 shows one rear upright instrument support with elements spaced far enough apart.

Rear upright instrument support 50 (FIGS. 2 and 3-FIG. 3 also shows instruments) has first and second, spaced-apart standing members 52 and 54 with at least one gap between the standing members. The rear upright instrument support in FIGS. 1 and 2 have two gaps 56 and 58 with center plate 60 between the gaps. The bottom of legs 62 and 64 contact the surface of base 14. The bottoms may be automatically laser welded to base 14 though the system can use other modes of fastening. The drawings do not show the structure at the bottom of the legs for attaching the legs to base 14.

In FIG. 2, each rear upright instrument support has rear-facing panels 68 and 70. Each panel has a groove 72, 74.

FIG. 8 shows how the described rear upright instrument support 50 holds the handle ends of instrument such as forceps 150. The structure around finger holes 152 and 154 of the bottom forceps rests at the bottom of grooves 72 and 74, and shanks 156 and 158 rest of the bottom of gaps 56 and 58. Forceps 160 is above forceps 150, and the depths of the gaps and grooves allow for additional instruments. The structure holds the instruments securely so they project forward from the support without moving much left to right.

The rear upright instrument supports may position the instruments they hold with the jaws of forceps or the blades of scissors apart (see FIG. 3) for sterilization of the jaws or blades.

FIG. 6 shows a different rear upright instrument support 80 for holding the handle end of an instrument such as forceps 170. Spaced-apart standing members 82 and 84 extend into curved sections 86 and 88. The structure around finger holes 172 and 174 may conform to the curved sections. The curved section may have offset ends to help placing the instrument's finger openings on the curved section.

Legs 92 and 94 of rear upright instrument support 80 are shorter than the legs of support 50 (FIG. 8). Support 80 holds more instruments, so the shorter legs position the top instrument at about the same height for both supports.

Standing members 82 and 84 have no center plate like plate 60 in FIG. 8. Thus, gap 66 in FIG. 6 is uninterrupted and wider.

FIG. 7 shows a third rear upright instrument support 100. It may be formed from bent sheet stainless steel. It has a front, upright wall 102 with two cutouts 104 and 106 and two upright extensions 108 and 110, which extend from base 112. Rear wall 114 extends upward from the base, and it bends rearward at 116 and then upward to 118 to form a shelf for the rear end of forceps. Spacer 120 keeps the rear of the forceps apart. The spacer and the inside of upright extensions 108 and 110 secure the rear of the forceps. Ledge 122 is a support surface of the bottom-most instrument.

When the management system is set to have a particular number of instruments, the rear upright instrument supports can be chosen to have corresponding distances for the instrument support surfaces above base 14. From that height, the distance of the top instrument can be determined. If the distance of all top instruments above the base is the same, the system can use rear upright instrument supports with the correct distance from the base to the surface supporting the bottom instrument.

Forward supports 130 (FIGS. 2-5) mount forward of each rear upright instrument support. For many forceps or scissors, the forward supports are positioned at or near where the hinge will be when the instruments mount in the rear upright instrument supports.

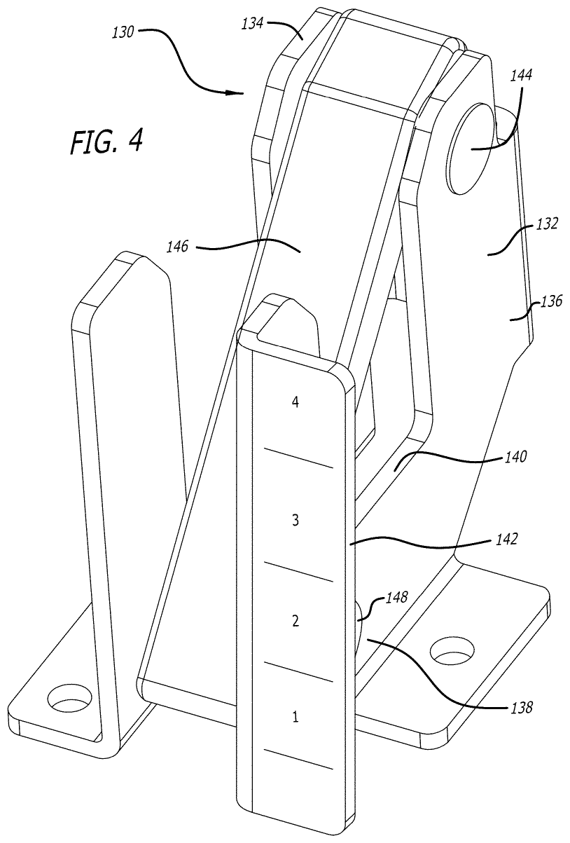

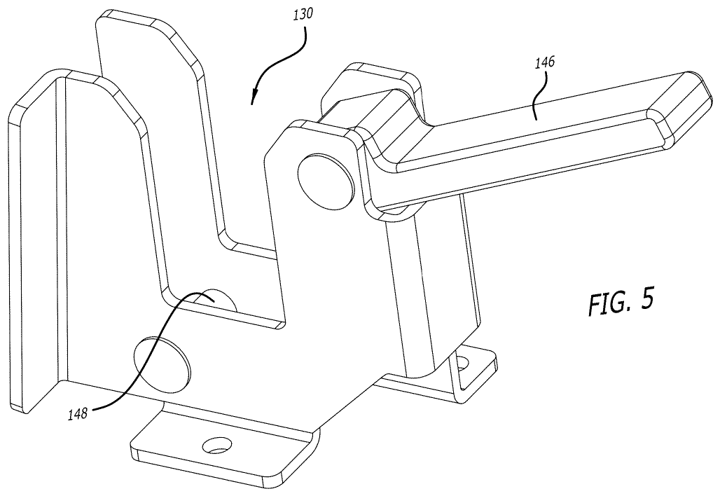

Forward support 130 in FIGS. 4 and 5 has first and second spaced-apart members 132 and 134. Having two members spaced-apart adds rigidity to the forward support, but a single member could be acceptable. Upright member 132 has first and second sides 136 and 138 with platform 140 between the sides. The platform is the surface on which the bottom surgical instrument rests.

When a second member 134 is used, it has a shape like the shape of first member 132. Both members are welded to base 14.

Gauge 142 extends upward for the second side of the first member. Pin or hinge 144 mounts through the first side of the platform, and the proximal end of bar 146 mounts for pivoting on the hinge. The bar's distal end extends near the gauge. The gauge is designed so that bar 146 will be horizontal and aligned with the “4” on the gauge when the system holds four instruments. If four instruments are removed, the bar drops below the “1” and contacts stop 148.

Bar 146 can pivot to and beyond vertical, see FIG. 5, to remove instruments and to return them to forward supports 130. During surgery, the bar should not remain in the FIG. 5 position. If an instrument is removed, the bar should be pivoted back-counter-clockwise in FIG. 5. If instruments are being reloaded after surgery, the bar can remain in the FIG. 5 position until the forward support is full. If the bar is above horizontal after it is fully loaded, it indicates that the support is overloaded.

The description is illustrative, not limiting and is for example only. Although this application shows and describes examples, those having ordinary skill in the art will find it apparent they can make changes, changes, or alterations. Many examples involve specific combinations of method acts or system elements, but those acts and those elements may be combined in other ways to meet the same goals. Acts, elements, and features discussed only with one embodiment are not intended to be excluded from a similar role in other embodiments.

“Plurality” means two or more. A “set” of items may include one or more of such items. The terms “comprising,” “including,” “carrying,” “having,” “containing,” “involving,” and similar words in the written description or the claims are open-ended, i.e., each means, “including but not limited to.” Only the transitional phrases “consisting of” and “consisting essentially of” are closed or semi-closed transitional phrases regarding claims. The ordinal terms like “first,” “second,” “third,” etc., in the claims don't by themselves connote any priority, precedence, or order of one claim element over another or the temporal order in which acts of a method are performed. Instead, they merely are labels to distinguish one claim element having a certain name from another element having a same name (but for the ordinal term's use). Alternatives like “or” include one or any combination of the listed items.

I claim:

1. A management system for holding instruments of which the instruments include a handle part and a part forward of the handle part, the management system comprising:
a base,
a rear upright instrument support mounted on the base and a forward upright instrument support mounted on the base and spaced from the rear upright instrument support,
the rear upright instrument support configured for holding the handle part of a plurality of instruments in an aligned stack from a bottom most instrument to a topmost instrument in which each handle part of the aligned stack of instruments is in contact with the handle part of at least one adjacent instrument,
the forward upright instrument support comprises at least one upright instrument support, each upright instrument support having at least a first side and a first platform extending from the first side, the first platform being positioned to support a part of each instrument in an aligned stack spaced from the handle part,
the management system further comprising a hinge extending from one upright instrument support, a bar having a proximal end pivotally mounted on the hinge and positioned to contact the topmost instrument when instruments are on the first platform, the bar having a distal end spaced from the first end,
wherein the bar being adapted to pivot under gravity in a path between a first position in contact with the topmost instrument in a first stack of instruments to a second position in contact with the topmost instrument of a second stack of instruments in which the first stack of instruments and the second stack of instruments have different numbers of instruments.

2. The management system of claim 1 further comprising a stop attached to the first side of at least one upright instrument support and positioned in the path of the bar to stop the bar from pivoting to a third position.

3. The management system of claim 1 further comprising a gauge positioned near the distal end of the bar, the gauge having indicia indicating the number of instruments in the forward upright instrument support.

4. The management system of claim 1 wherein the forward upright instrument support comprises a first upright instrument support and a second upright instrument support spaced from the first upright instrument support, the second upright instrument support having at least a first side and a second platform extending from the first side, the first platform and the second platform being positioned to support a part of each instrument in an aligned stack.

5. The management system of claim 4, wherein the first platform and the second platform are parallel to each other.

6. The management system of claim 4 wherein the hinge attaches to the first upright instrument support and the second upright instrument support.

7. The management system of claim 4 wherein the first and second upright instrument supports are spaced far enough apart to allow the bar to pivot between the first and second upright instrument supports.

8. The management system of claim 1, further comprising a gauge extending upward from the forward upright instrument support, the bar having a distal end extending near the gauge, wherein the gauge has spaced-apart indicators indicative of the number of instruments remaining in the rear upright instrument support.

9. The management system of claim 1, wherein the bar is adapted to pivot in the path between the first and second position to contact the topmost instruments when an instrument is removed from the stack of instruments.

10. The management system of claim 1, wherein the forward upright instrument support is positioned at or near where the hinge will be when the instruments are in the forward upright instrument supports.

11. A management system for holding instruments comprising:
a base,
at least one, rear upright instrument support extending upward from the base, each rear upright instrument support having at least one surface sized and positioned to receive a first part of a stack of instruments in which each first part of the stack of instruments is in contact with the first part of at least one adjacent instrument,
a forward support attached to the base and having at least one forward, upright instrument support, the at least one forward, upright instrument support having a first platform on which a second part of an instrument can contact and at least a first side extending upward from the first platform,
a gauge adjacent the at least one forward, upright instrument support, a hinge on at least one forward, upright instrument support and spaced from the platform,
a bar having a proximal end pivotally mounted on the hinge and adapted to pivot in a path between a first position and a second position, the bar having a distal end extending near the gauge, the bar being adapted to pivot in the path between the first and second positions when an instrument is removed or added to the forward support.

12. The management system of claim 11, wherein the forward support comprises at least a first and a second spaced apart upright instrument support.

13. The management system of claim 12, wherein the hinge extends between a first and a second spaced apart upright instrument supports.

14. The management system of claim 11, wherein the first upright instrument support has first and second sides, the first platform extending between the first and second sides of the first upright instrument support, and a second platform extending between the first and second sides of the second upright instrument support.

15. The management system of claim 14, wherein the first and second platforms are parallel to each other.

16. The management system of claim 11 further comprising a stop attached to at least one, forward upright instrument support in the path of the bar, the stop stopping the bar from pivoting to a third position.

17. The management system of claim 11, wherein the stop extends between the first and second upright instruments.

18. The management system of claim 11, wherein the gauge has spaced-apart indicators indicative of the number of instruments remaining in the rear upright instrument support.

19. The management system of claim 11, wherein the bar is adapted to pivot in the path between the first and second position to contact the topmost instruments when an instrument is removed from the stack of instruments.

20. A method of managing instruments comprising:
mounting at least one rear upright instrument support on a base, the rear upright instrument support having a rear support surface with a height from the base,
aligning a forward, upright instrument support with each forward upright instrument support, the forward, upright instrument support having a forward support surface and having a height from the base equal to the height of the forward support surface,
moving a bar from a position covering the forward support surface to a position exposing the forward support surface,
placing enough instruments on the rear support surface and on the forward support surface when the bar is positioned to expose the forward support surface and positioning the bar to a horizontal position in contact with an instrument.
